(19) United States
(12) Patent Application Publication
(10) Pub. No.: US 2025/0221625 A1
(43) Pub. Date: Jul. 10, 2025

(54) IMAGE DIAGNOSTIC SYSTEM, IMAGE DIAGNOSTIC METHOD, AND STORAGE MEDIUM

(71) Applicant: TERUMO KABUSHIKI KAISHA, Tokyo (JP)

(72) Inventor: Takanori TOMINAGA, Hadano Kanagawa (JP)

(21) Appl. No.: 19/092,963

(22) Filed: Mar. 27, 2025

Related U.S. Application Data
(63) Continuation of application No. PCT/JP2023/034949, filed on Sep. 26, 2023.

(30) Foreign Application Priority Data
Sep. 28, 2022 (JP) 2022-155192

(51) Int. Cl. A61B 5/00; A61B 5/02; A61B 8/12; G06T 7/00; G06T 7/12; G06T 7/62; G06T 11/20

(52) U.S. Cl. CPC A61B 5/0084; A61B 5/0066; A61B 5/02007; A61B 5/743; A61B 5/7435; A61B 5/7475; G06T 7/0012; G06T 7/12; G06T 7/62; G06T 11/206; A61B 8/12; G06T 2200/24; G06T 2207/10072; G06T 2207/20092; G06T 2207/20212; G06T 2207/30101

(57) ABSTRACT

An image diagnostic system includes a catheter insertable into a luminal organ and including imaging devices, a display, and a processor configured to execute a program to perform: generating tomographic images of the organ based on signals that are output from the devices, combining the images to generate a first tomographic image, calculating one or more distributions of values each indicating an anatomical feature of the organ from the first tomographic image, selecting at least one of the distributions and specifying one or more first portions of the organ that are located along a longitudinal direction of the organ and at which the distribution of values meets one or more conditions, and controlling the display to display: one or more graphs each illustrating a corresponding one of the calculated distributions, and one or more first graphics on the graphs at locations corresponding to the specified first portions.

### CROSS-REFERENCE TO RELATED APPLICATION

This application is a continuation of International Patent Application No. PCT/JP2023/034949 filed Sep. 26, 2023, which is based upon and claims the benefit of priority from Japanese Patent Application No. 2022-155192, filed Sep. 28, 2022, the entire contents of which are incorporated herein by reference.

### BACKGROUND

### Technical Field

Embodiments described herein relate to an image diagnostic system, an image diagnostic method, and a storage medium.

### Related Art

Medical catheters are used for diagnosis and treatment of lesions present in luminal organs such as blood vessels. An ultrasonic sensor or an optical sensor is provided in a medical catheter that is insertable into an organ, and images generated based on the signals obtained from the sensor are used for diagnosis.

In particular, image diagnosis of blood vessels is indispensable for safely and reliably performing operations such as percutaneous coronary intervention (PCI). For this reason, intravascular imaging techniques using medical catheters such as intravascular ultrasound (IVUS), optical coherence tomography (OCT)/optical frequency domain imaging (OFDI), and the like are widely used in addition to angiography techniques for performing imaging from outside the body using contrast agents.

Medical workers such as medical doctors perform diagnosis and treatment by grasping the states of luminal organs with reference to medical images based on these imaging techniques. There have been proposed various techniques for not only displaying medical images on a display but also generating and displaying information for assisting medical workers to interpret such medical images by image processing or computation.

### SUMMARY

After interpreting the medical images and grasping the anatomical features of the luminal organ of the patient and the state of the lesion, the medical worker performs treatment of pushing and expanding the occluded luminal organ itself using a balloon provided at the distal end of the medical catheter for treatment or placing a stent inside the luminal organ. At this time, it is desired to output information such that the range of the lesion and the state of the surrounding luminal organ can be accurately grasped based on the medical image.

Embodiments of the present disclosure provide an image diagnostic system, an image diagnostic method, and a storage medium capable of displaying appropriate information necessary for medical diagnosis based on a medical image.

In one embodiment, an image diagnostic system comprises: a catheter insertable into a luminal organ and including a plurality of imaging devices; a display; a memory; and a processor configured to execute a program that is stored in the memory to perform the steps of: generating tomographic images of the luminal organ based on signals that are output from the imaging devices, combining the tomographic images to generate a first tomographic image, calculating one or more distributions of values each indicating an anatomical feature of the luminal organ from the first tomographic image, selecting at least one of the distributions and specifying one or more first portions of the luminal organ that are located along a longitudinal direction of the luminal organ and at which the distribution of values of the selected distribution meets one or more conditions, and controlling the display to display: one or more graphs each illustrating a corresponding one of the calculated distributions, and one or more first graphics on the graphs at locations corresponding to the specified first portions.

According to the present disclosure, it is possible to realize display for accurately grasping a range satisfying a setting condition in a luminal organ with respect to data indicating an anatomical feature of a luminal organ.

### BRIEF DESCRIPTION OF DRAWINGS

FIG. 1 is a schematic diagram of an image diagnostic system.

FIG. 2 is an explanatory diagram illustrating an operation using a catheter.

FIG. 3 is a block diagram illustrating a configuration of an image processing apparatus.

FIG. 4 is a schematic diagram of a segmentation model.

FIG. 5 is a diagram illustrating a detected boundary.

FIG. 6 is a flowchart illustrating a part of a process performed by the image processing apparatus.

FIG. 7 is a flowchart illustrating another part of the process performed by the image processing apparatus.

FIG. 8 illustrates an example of a screen displayed on a display apparatus.

FIG. 9 illustrates an example of a list screen.

FIG. 10 illustrates another example of a screen displayed on the display apparatus.

FIG. 11 is a flowchart illustrating a part of a process performed by an image processing apparatus according to a second embodiment.

FIG. 12 is a flowchart illustrating another part of a process performed by the image processing apparatus according to the second embodiment.

FIG. 13 illustrates an example of a screen displayed on a display apparatus according to the second embodiment.

FIG. 14 illustrates another example of a screen displayed on the display apparatus according to the second embodiment.

FIG. 15 illustrates another example of a screen displayed on the display apparatus according to the second embodiment.

FIG. 16 is a flowchart illustrating a part of a process performed by an image processing apparatus according to a third embodiment.

FIG. 17 is a flowchart illustrating another part of a process performed by the image processing apparatus according to the third embodiment.

FIG. 18 illustrates an example of a screen displayed on a display apparatus according to the third embodiment.

FIG. 19 is a flowchart illustrating a part of a process performed by an image processing apparatus according to a fourth embodiment.

FIG. 20 is a flowchart illustrating another part of the process performed by the image processing apparatus according to the fourth embodiment.

FIG. 21 illustrates an example of a screen displayed on a display apparatus according to the fourth embodiment.

### DETAILED DESCRIPTION

Embodiments of the present invention will be described below with reference to the drawings. In the following embodiments, information processing for a blood vessel will be described as an example of a luminal organ, but it is a matter of course that the luminal organ is not limited to the blood vessel.

### First Embodiment

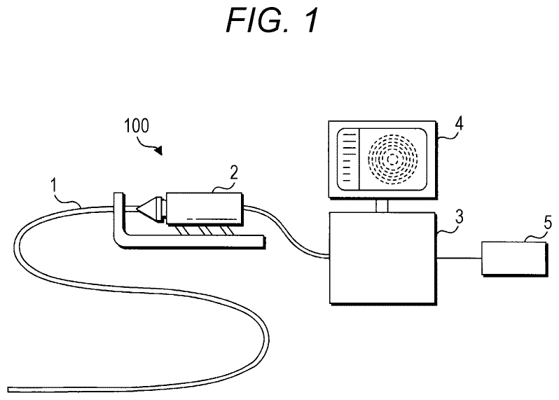

FIG. 1 is a schematic diagram of an image diagnostic system 100. The image diagnostic system 100 includes a catheter 1, a motor drive unit (MDU) 2, an image processing apparatus 3, a display apparatus 4, and an input apparatus 5.

The catheter 1 is a medical flexible tube. The catheter 1 is called an imaging catheter in which an imaging device 11 is provided at a distal end portion and is rotated in a circumferential direction by driving from a proximal end. The imaging device 11 of the catheter 1 is an ultrasonic probe including an ultrasonic transducer and an ultrasonic sensor in the case of the IVUS method. In the case of OFDI, the imaging device 11 may be an OFDI device including a near-infrared laser, a near-infrared sensor, and the like, or may be a device including an optical element having a lens function and a reflection function at the distal end, and may have a structure of guiding light to a light source or an optical sensor connected via an optical fiber. The imaging device 11 may include both an ultrasonic probe for IVUS and an optical element for OFDI. In this case, the catheter 1 is referred to as a dual type catheter. As the imaging device 11, other devices using electromagnetic waves of other wavelengths such as visible light may be used.

The MDU 2 is a drive apparatus attached to the proximal end of the catheter 1, and controls the operation of the catheter 1 by driving an internal motor according to the operation of the inspection operator.

The image processing apparatus 3 generates a plurality of medical images such as tomographic images of a blood vessel based on the signals output from the imaging device 11 of the catheter 1. Details of the configuration of the image processing apparatus 3 will be described later.

As the display apparatus 4, a liquid crystal display panel, an organic electro luminescence (EL) display panel, or the like is used. The display apparatus 4 displays a medical image generated by the image processing apparatus 3 and information regarding the medical image.

The input apparatus 5 is an input interface that receives an operation on the image processing apparatus 3. The input apparatus 5 may be a keyboard, a mouse, or the like, or may be a touch panel, a software key, a hardware key, or the like built in the display apparatus 4. The input apparatus 5 may receive an operation based on voice input. In this case, the input apparatus 5 uses a microphone and a speech recognition engine.

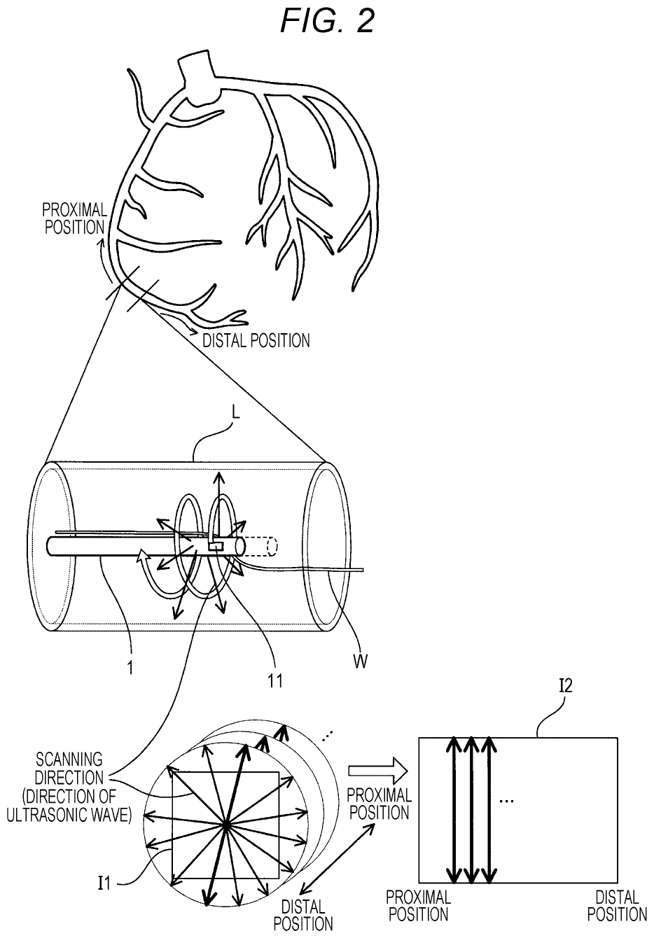

FIG. 2 is an explanatory diagram illustrating am operation using the catheter 1. In FIG. 2, the catheter 1 is inserted into a tubular blood vessel L (e.g., a coronary artery) along a guide wire W inserted by an inspection operator. In the enlarged view of the blood vessel L in FIG. 2, the right portion corresponds to the distal position from the insertion position of the catheter 1 and the guide wire W, and the left portion corresponds to the proximal position.

The catheter 1 moves from the distal position to the proximal position in the blood vessel L and scans the inside of the blood vessel in a spiral manner by the imaging device 11 while rotating in the circumferential direction by driving the MDU 2 as indicated by an arrow in the figure.

In the image diagnostic system 100 of the present embodiment, the image processing apparatus 3 acquires a signal for each scan output from the imaging device 11 of the catheter 1. One scan is to emit a detection wave from the imaging device 11 in the radial direction and detect reflected light, and is performed in a spiral manner. The image processing apparatus 3 generates a tomographic image (i.e., a cross-sectional image) obtained by performing polar coordinate transformation on a signal for each scan every 360 degrees (I1 in FIG. 2). The tomographic image I1 is also referred to as a frame image. The reference point (i.e., the center) of the tomographic image I1 corresponds to the range (not imaged) of the catheter 1. The image processing apparatus 3 further generates a longitudinal cross-sectional image in which the catheter 1 arranges pixel values on a straight line passing through the reference point of the tomographic image I1 along the length direction or longitudinal direction of the blood vessel (I2 in FIG. 2). The image processing apparatus 3 calculates data indicating anatomical features of a blood vessel based on the obtained tomographic image I1 and longitudinal image I2, and outputs the tomographic image I1 or the longitudinal image I2 and the calculated data so that a doctor, an inspection operator, or other medical workers can visually recognize the tomographic image I1 or the longitudinal image I2 and the calculated data.

In the image diagnostic system 100 according to the present disclosure, the image processing apparatus 3 performs image processing on the tomographic image I1 or the longitudinal image I2 to output the anatomical features in the blood vessel and the state of the lesion in an easily understandable manner. Specifically, the image processing apparatus 3 performs output so as to emphasize the range in which the anatomical features in the blood vessel satisfy the setting condition. Hereinafter, the output processing by the image processing apparatus 3 will be described in detail.

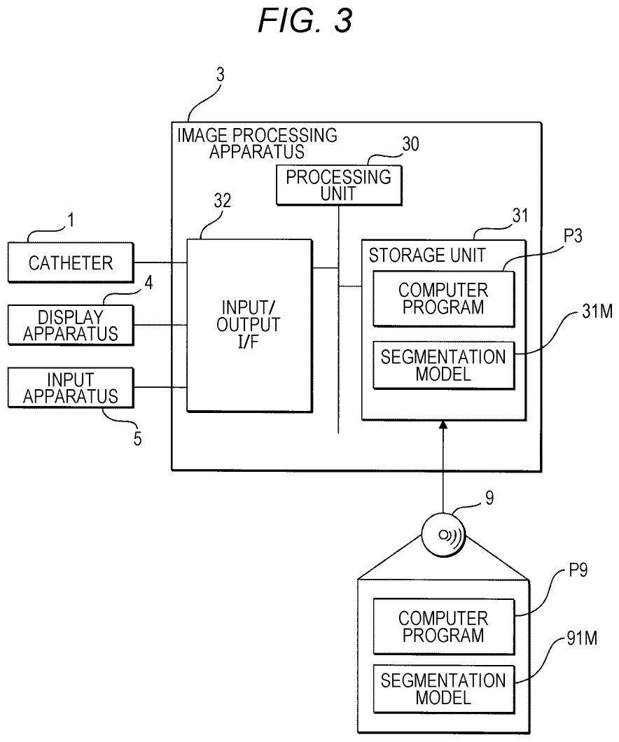

FIG. 3 is a block diagram illustrating a configuration of the image processing apparatus 3. The image processing apparatus 3 includes a processing unit 30, a storage unit 31, and an input/output I/F 32.

The processing unit 30 includes one or more processors such as central processing units (CPUs), micro-processing units (MPUs), graphics processing units (GPUs), general-purpose computing on graphics processing units (GPGPU), tensor processing units (TPUs), and the like. The processing unit 30 incorporates a memory such as a random access memory (RAM), and executes computation based on a computer program P3 stored in the storage unit 31 while storing data generated during processing in the non-temporary storage medium.

The storage unit 31 is a non-volatile storage medium such as a hard disk or a flash memory. The storage unit 31 stores the computer program P3 read by the processing unit 30, setting data (described later), and the like. In addition, the storage unit 31 stores a trained segmentation model 31M.

The computer program P3 and the segmentation model 31M may be obtained by reading a computer program P9 and a segmentation model 91M stored in a non-temporary storage medium 9 via the input/output I/F 32 and replicating the computer program P9 and the segmentation model 91M. The computer program P3 and the segmentation model 31M may be those distributed by a remote server apparatus and acquired by the image processing apparatus 3 via a communication unit (not illustrated) and stored in the storage unit 31.

The input/output I/F 32 is an interface circuit to which the catheter 1, the display apparatus 4, and the input apparatus 5 are connected. The processing unit 30 acquires a signal (i.e., digital data) output from the imaging device 11 via the input/output I/F 32. The processing unit 30 outputs screen data of a screen including the generated tomographic image I1 and/or longitudinal image I2 to the display apparatus 4 via the input/output I/F 32. The processing unit 30 receives operation information input to the input apparatus 5 via the input/output I/F 32.

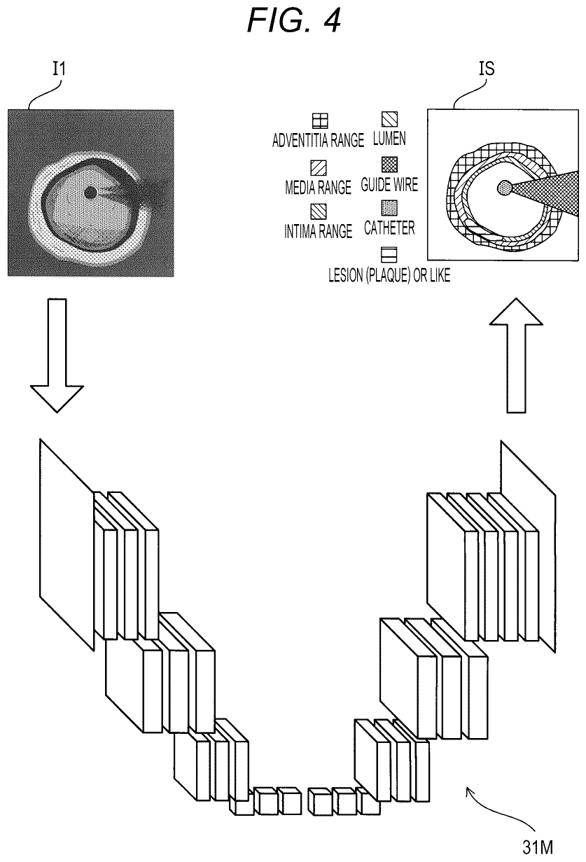

FIG. 4 is a schematic diagram of the segmentation model 31M. The segmentation model 31M is a computer model trained to output an image indicating a region of one or a plurality of target objects present in an image when the image is input. The segmentation model 31M is, for example, a model that performs semantic segmentation. The segmentation model 31M is designed to output, for each pixel in the input image, an image tagged with data indicating which target object is present in each pixel of the range present.

As illustrated in FIG. 4, for example, a so-called U-net in which a convolution layer, a pooling layer, an upsampling layer, and a softmax layer are arranged as a target is used as the segmentation model 31M. The segmentation model 31M outputs the tag image IS when the tomographic image I1 created by the signal from the catheter 1 is input. In the tag image IS, the lumen range of the blood vessel, the membrane range corresponding to between the lumen boundary of the blood vessel including the media of the blood vessel and the blood vessel boundary, the range in which the guide wire W and its reverberation are present, and the range corresponding to the catheter 1 are tagged with different pixel values (indicated by different types of hatching and blank in FIG. 4) to the pixels at the positions. The segmentation model 31M further identifies a portion of a fibrous plaque formed in a blood vessel, a portion of a lipid plaque, a portion of a calcified plaque, and the like.

For the segmentation model 31M, the semantic segmentation and the U-net have been exemplified as described above, but the segmentation model 31M is not limited thereto. In addition, the segmentation model 31M may be a model that realizes individual recognition processing by instance segmentation or the like. The segmentation model 31M is not limited to the U-net base, and a model based on SegNet, R-CNN, an integrated model with other edge extraction processing, or the like may be used.

The processing unit 30 identifies the blood (or the lumen range), the intima range, the media range, and the adventitia range of the blood vessel present in the tomographic image I1 by the pixel value in the tag image IS obtained by inputting the tomographic image I1 to the segmentation model 31M and the coordinates in the image. The processing unit 30 can detect the lumen boundary and the blood vessel boundary of the blood vessel present in the tomographic image I1 by identifying the range of the blood vessel. The blood vessel boundary is strictly the external elastic membrane (EEM) between the media and the adventitia of the blood vessel.

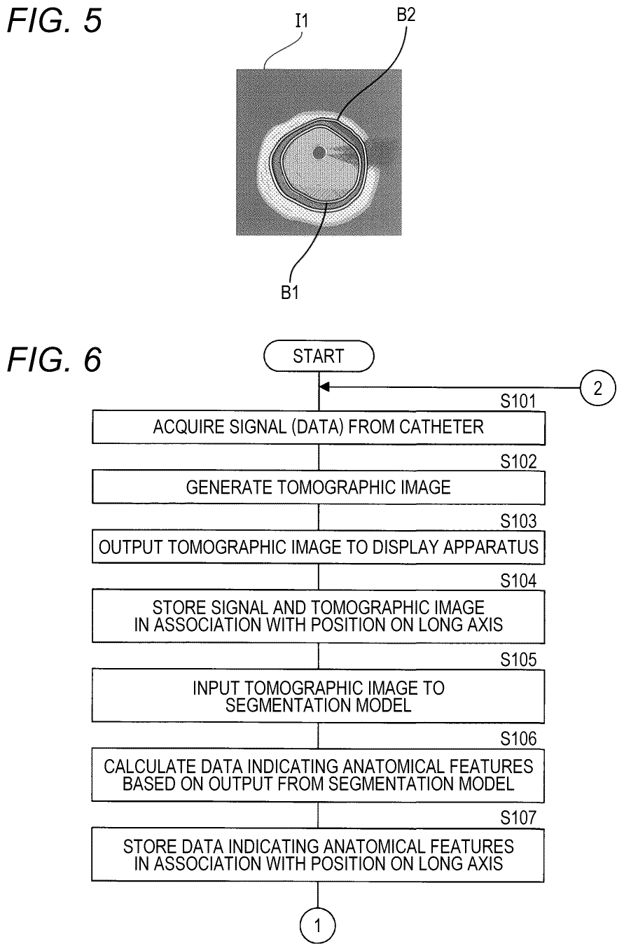

FIG. 5 is a diagram illustrating a detected boundary or contour. FIG. 5 illustrates a state of a blood vessel in which a curve B1 indicating the lumen boundary and a curve B2 indicating the blood vessel boundary obtained based on the output from the segmentation model 31M are displayed in a superimposed manner on the tomographic image I1 illustrated in FIG. 4.

In the present embodiment, the processing unit 30 specifies the lumen boundary of the lumen range of the blood vessel from each range identified with respect to the tomographic image I1 as illustrated in FIG. 5, and calculates numerical values such as the maximum diameter, the minimum diameter, and the average inner diameter inside the lumen boundary. Furthermore, the processing unit 30 calculates the ratio of the cross-sectional area to the area inside the blood vessel boundary (hereinafter, referred to as plaque burden) from the identification result of the fibrous plaque range, the lipid plaque range, or the calcified plaque range by the segmentation model 31M. The image processing apparatus 3 of the present disclosure further executes a process of graphically outputting the distribution of the average lumen diameter with respect to the position in the longitudinal direction of the blood vessel and the distribution of the plaque burden, and identifiably outputting the range satisfying the setting condition in the graph.

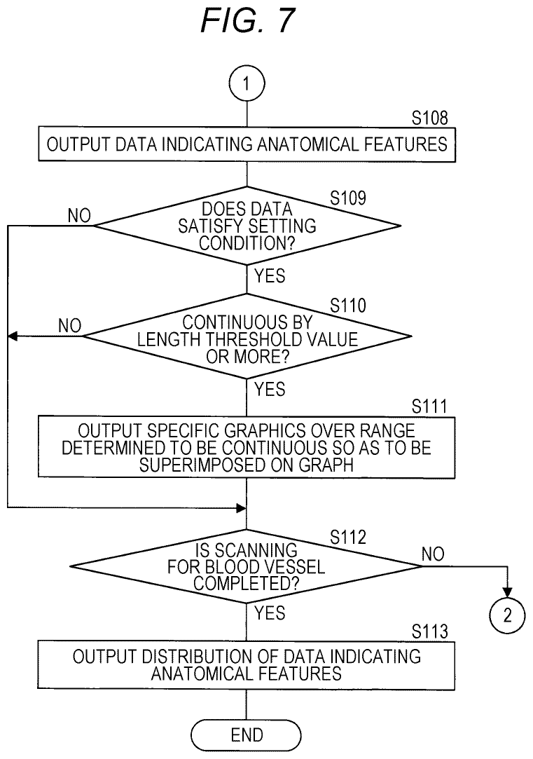

A process performed by the image processing apparatus 3 will be described with reference to a flowchart. FIGS. 6 and 7 are flowcharts illustrating an example of a process performed by the image processing apparatus 3. When a signal is output from the imaging device 11 of the catheter 1, the processing unit 30 of the image processing apparatus 3 starts the following process.

The processing unit 30 performs polar coordinate transformation (e.g., inverse transformation) on signals arranged in a rectangle to generate the tomographic image I1 (S102) each time a predetermined amount (for example, 360 degrees) of signals from the imaging device 11 of the catheter 1 is acquired (S101). The processing unit 30 outputs the generated tomographic image I1 so that the generated tomographic image I1 can be displayed in real time in a screen displayed on the display apparatus 4 (S103). The processing unit 30 stores the signal data acquired in S101 and the tomographic image I1 in the storage unit 31 in association with the position on the longitudinal axis of the blood vessel (S104).

The processing unit 30 inputs the tomographic image I1 to the segmentation model 31M (S105).

On the basis of the tag image IS output from the segmentation model 31M, the processing unit 30 calculates data indicating anatomical features including the maximum value, the minimum diameter, the average inner diameter, and the plaque burden in the range inside from the lumen boundary in the tomographic image I1 (S106). In S106, the processing unit 30 may calculate the maximum diameter, the minimum diameter, and the average diameter in the range inside from the blood vessel boundary.

The processing unit 30 stores the data (e.g., the average inner diameter inside the lumen boundary, plaque burden, and the like) indicating the anatomical features calculated in S106 in the storage unit 31 in association with the position on the longitudinal axis of the blood vessel corresponding to the tomographic image I1 (S107).

The processing unit 30 outputs the data indicating the anatomical feature calculated in S107 as a graph in the screen being displayed on the display apparatus 4 (S108). In S108, the processing unit 30 outputs a graph indicating a transition of data indicating anatomical features with respect to the longitudinal direction. In S108, the processing unit 30 may also display the numerical value itself of the data.

The processing unit 30 determines whether the data indicating the anatomical features calculated in S107 satisfies a setting condition included in the setting data stored in the storage unit 31 (S109). In a case where the plaque burden is targeted in S109, the setting condition is that the plaque burden is equal to or more than a threshold value of the ratio. When the average lumen diameter is used as a target in S109, the setting condition is that the average lumen diameter is less than a threshold value of the lumen diameter.

When it is determined that the data indicating the anatomical features satisfies the setting condition (S109: YES), the processing unit 30 determines whether the data determined to satisfy the setting condition is continuous in the longitudinal direction by a length threshold value or more included in the setting data (S110). The threshold value of the length included in the setting data in S110 may be set to a value different depending on whether the target is plaque burden or the average lumen diameter.

When it is determined in S110 that the data is continuous (S110: YES), the processing unit 30 outputs graphics of specific colors, patterns, and the like over the range of positions on the longitudinal axis determined to be continuous so as to be superimposed on the graph output in S108 (S111).

The processing unit 30 determines whether scanning of the catheter 1 by the imaging device 11 has been completed (S112). When it is determined that the scanning is not completed (S112: NO), the processing unit 30 returns the process to S101 and generates the next tomographic image I1.

When it is determined that the scanning is completed (S112: YES), the processing unit 30 displays again the distribution of the data indicating the anatomical features with respect to the entire scanned blood vessel in the longitudinal direction (S113), and ends the process.

When it is determined in S109 that the setting condition is not satisfied (S109: NO), the processing unit 30 advances the processing directly to S112. When it is determined in S110 that the data does not continue for the length threshold value or more (S110: NO), the processing unit 30 advances the process directly to S112.

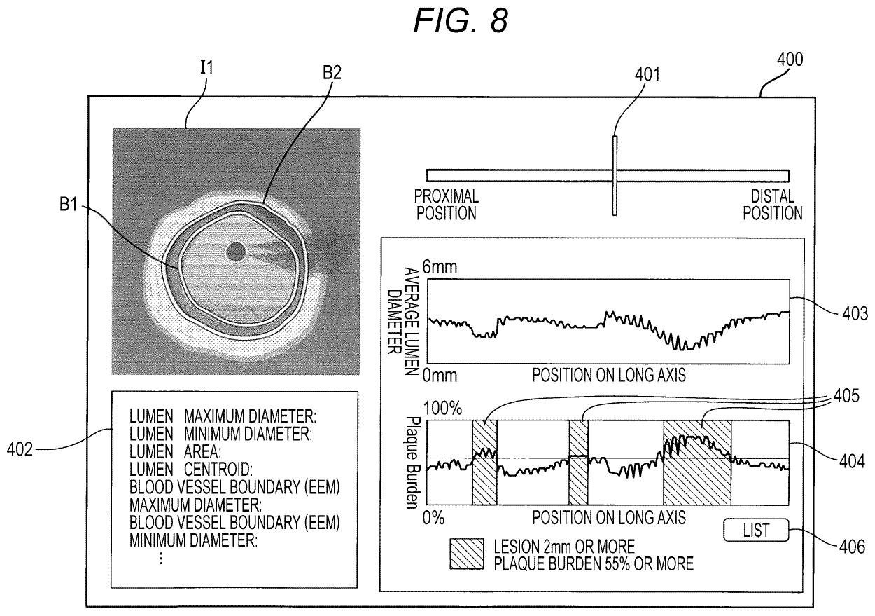

The information output by the process illustrated in FIGS. 6 and 7 will be described with specific examples. FIG. 8 illustrates an example of a screen 400 displayed on the display apparatus 4. Note that the screen 400 illustrated in FIG. 8 illustrates a screen in a state where scanning from the proximal position to the distal position of the blood vessel is completed.

The screen 400 includes a cursor 401 indicating a position on the longitudinal axis of the blood vessel corresponding to the tomographic image I1 to be displayed, and a tomographic image I1 generated based on a signal obtained at the position. In the tomographic image I1, a curve B1 indicating the lumen boundary identified by the processing of the image processing apparatus 3 and a curve B2 indicating the blood vessel boundary are displayed in a superimposed manner. The screen 400 includes a data field 402 that displays numerical values of data indicating anatomical features calculated by image processing on the tomographic image I1.

The screen 400 includes graphs 403 and 404 illustrating the distribution of data indicative of anatomical features relative to locations on the longitudinal axis of the blood vessel. The graph 403 illustrates the distribution of the average lumen diameter with respect to the position on the longitudinal axis. The graph 404 illustrates the distribution of plaque burden with respect to the position on the longitudinal axis.

A graphic 405 is superimposed and displayed on the graph 404. The graphic 405 indicates a range in which a portion where the plaque burden is equal to or more than the threshold value of the ratio (here, 55%) is continuous in the longitudinal direction by 2 mm or more. The threshold value (=55%) of the ratio and the threshold value (=2 mm) of the length are stored in advance in the storage unit 31 as setting data. The inspection operator and other medical workers who visually recognize the graph 404 on which the graphic 405 is superimposed can grasp that the plaque burden is equal to or more than the threshold value of the ratio over a length of 2 mm or more in the blood vessel in the range in which the graphic 405 is displayed. This makes it possible to more accurately determine which size should be selected as the stent for expanding the high plaque burden range and how to select the balloon for placing the stent.

The screen 400 further includes a button 406 for displaying a list. When the operator or the medical worker selects the button 406 using the input apparatus 5, a list of outputs in a case where conditions (a ratio threshold value and a length threshold value) for displaying the graphic 405 are changed is displayed.

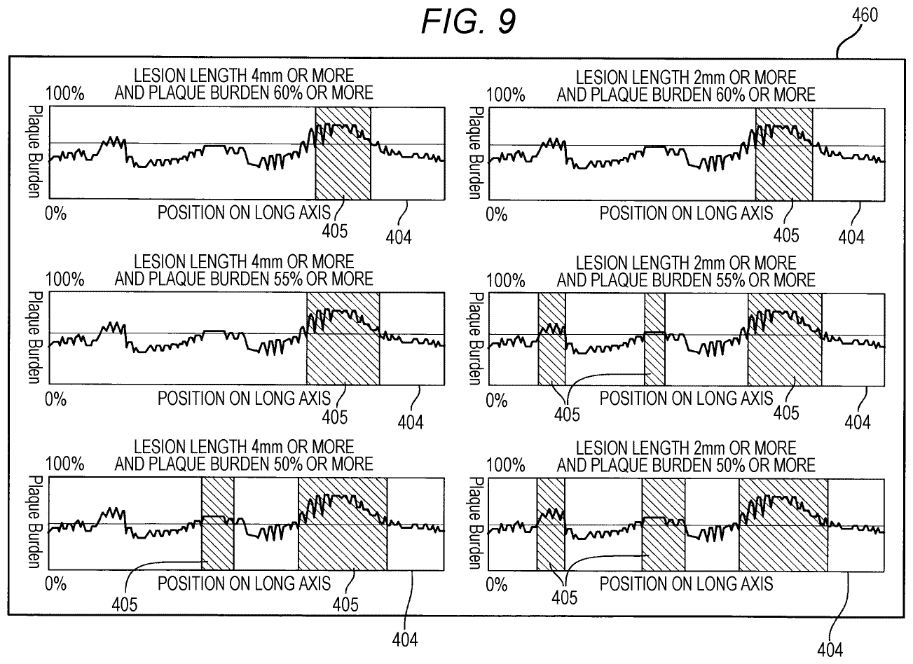

FIG. 9 illustrates an example of the list screen 460. The list screen 460 is displayed when the button 406 on the screen 400 is selected. The list screen 460 includes a plurality of graphs 404 in which the ratio threshold value and the length threshold value are changed. In FIG. 9, the list screen 460 illustrates graphs 404 and graphics 405 for each of six combinations of a case where the threshold value of the length to be emphasized as the lesion is set to two of 2 mm and 4 mm and a case where the threshold value of the ratio to plaque burden is set to three of 50%, 55%, and 60%. The graph itself illustrating the transition of plaque burden in the graph 404 is the same. Changes in the graphic 405 when the ratio threshold value and the length threshold value are changed are displayed as a list.

The inspection operator and the other medical workers who visually recognize the list screen 460 in FIG. 9 can confirm in the list that the way the graphic 405 is displayed differs depending on the conditions (i.e., the ratio threshold value and the length threshold value). As a result, it is possible to grasp the position of the part that requires reliable treatment as the lesion and the position of the part that is in the worst state.

In addition, the inspection operator and the other medical workers may select, from this list, which condition (i.e., the ratio threshold value and length threshold value) they want to display. For example, when any one of the ranges of the plurality of graphs 404 displayed on the list screen 460 is selected, the processing unit 30 changes the setting data to be displayed under the condition corresponding to the selected graph 404 and returns to the screen 400 of FIG. 8.

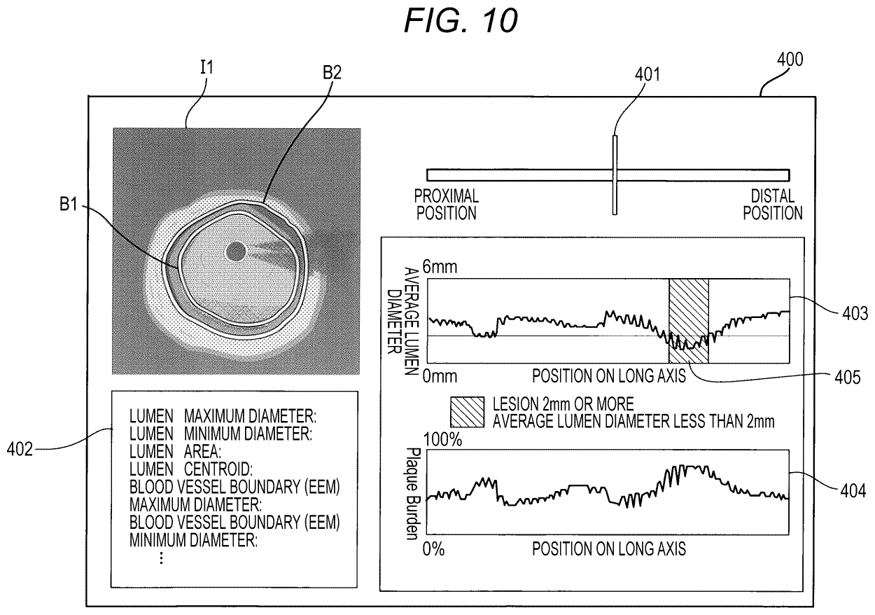

FIG. 10 illustrates another example of the screen 400 displayed on the display apparatus 4. FIG. 10 illustrates the screen 400 in a state where scanning from the proximal position to the distal position of the blood vessel is completed, similar to FIG. 8. Detailed description of common features between the screen 400 of FIG. 10 and the screen 400 illustrated in FIG. 8 is omitted. In the screen 400 of FIG. 10, a graphic 405 is superimposed and displayed on a graph 403 illustrating the distribution of the average lumen diameter. The graphic 405 illustrates a range in which a portion where the average lumen diameter is less than the threshold value of the lumen diameter (for example, 2 mm) is continuous in the longitudinal direction by 2 mm or more. The data of the threshold value (=2 mm) of the lumen diameter and the threshold value (=2 mm) of the length are stored in advance in the storage unit 31 as setting data. The inspection operator and the other medical workers who visually recognize the graph 403 on which the graphic 405 is superimposed can grasp that the average lumen diameter is less than the threshold value of the lumen diameter over a length of 2 mm or more in the blood vessel in the range in which the graphic 405 is displayed. This makes it possible to more accurately determine how to select a stent and a balloon for expanding the range where the average lumen diameter is small.

Also in the graph 403 of the average lumen diameter, it is possible to display a list in which the conditions (i.e., the threshold value of the lumen diameter and the threshold value of the length) are changed in the same manner as in FIG. 9.

### Second Embodiment

In a second embodiment, setting data can be changed while graphs are displayed on the screen 400. The configuration of an image diagnostic system 100 according to the second embodiment is similar to the configuration of the image diagnostic system 100 of the first embodiment except for details of a process performed by the processing unit 30 of the image processing apparatus 3 described below and contents of a screen to be displayed. Therefore, among the configurations of the image diagnostic system 100 of the second embodiment, the configurations common to the image diagnostic system 100 of the first embodiment are denoted by the same reference signs, and a detailed description thereof will be omitted.

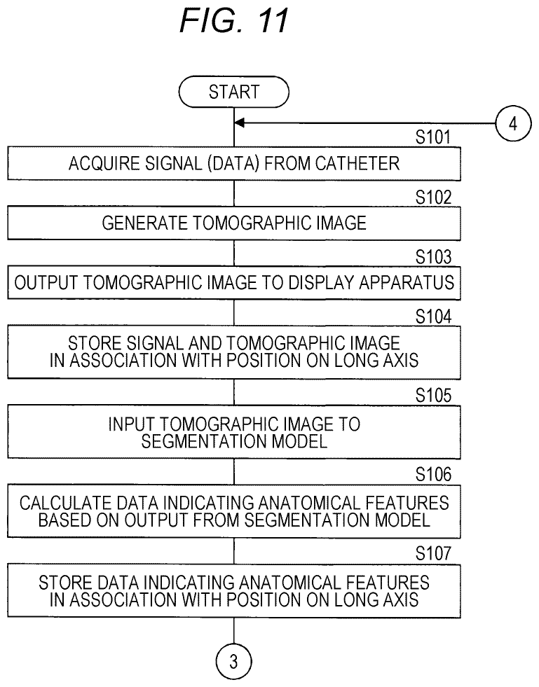

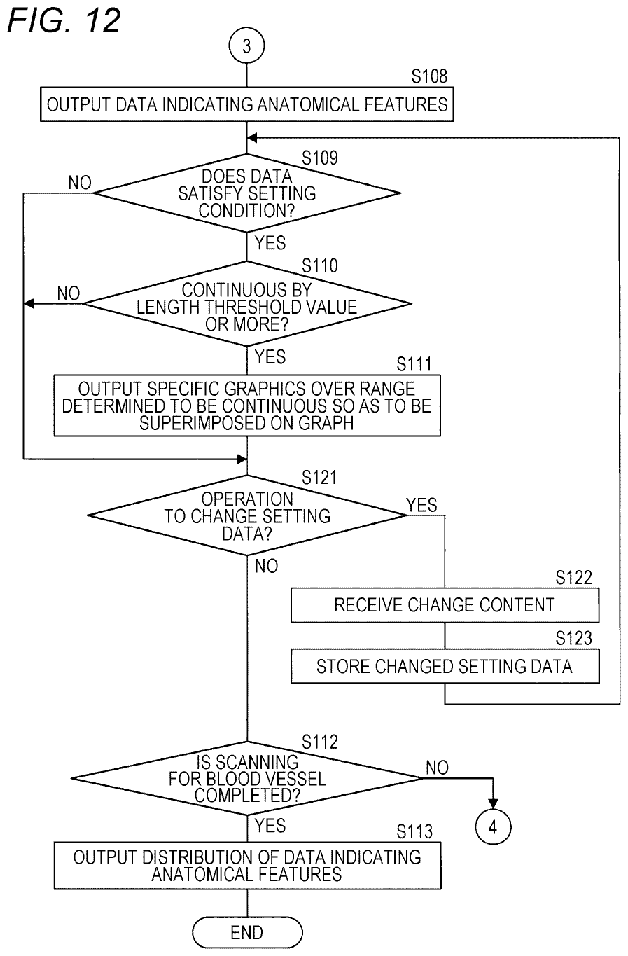

FIGS. 11 and 12 are flowcharts illustrating an example of the process performed by the image processing apparatus 3 according to the second embodiment. Among the steps illustrated in the flowcharts of FIGS. 11 and 12, the steps common to the process illustrated in the flowcharts of FIGS. 6 and 7 of the first embodiment are denoted by the same step numbers, and detailed description thereof is omitted.

In the second embodiment, the processing unit 30 of the image processing apparatus 3 determines whether an operation of changing setting data such as a setting condition or a length threshold value is performed on the screen on which the graph is output (S121) before determining whether scanning of the blood vessel is completed (S112).

When determining that the operation to change the setting data has not been performed (S121: NO), the processing unit 30 advances the process to S112.

When determining that the operation to change the setting data is performed (S121: YES), the processing unit 30 receives the change content (S122), and stores the changed setting data (i.e., a setting condition or length threshold value) in the storage unit 31 (S123). The processing unit 30 returns the process to S109, and based on the setting condition and the length threshold value included in the changed setting data, outputs graphics of a specific color and a mark to be superimposed on a range satisfying the setting condition continuously for a length threshold value or more (S111).

In S122, the processing unit 30 can receive a change not only for the plaque burden threshold value but also for each of the plurality of types of anatomical features. The processing unit 30 may receive a change in setting for both the ratio threshold value and the length threshold value.

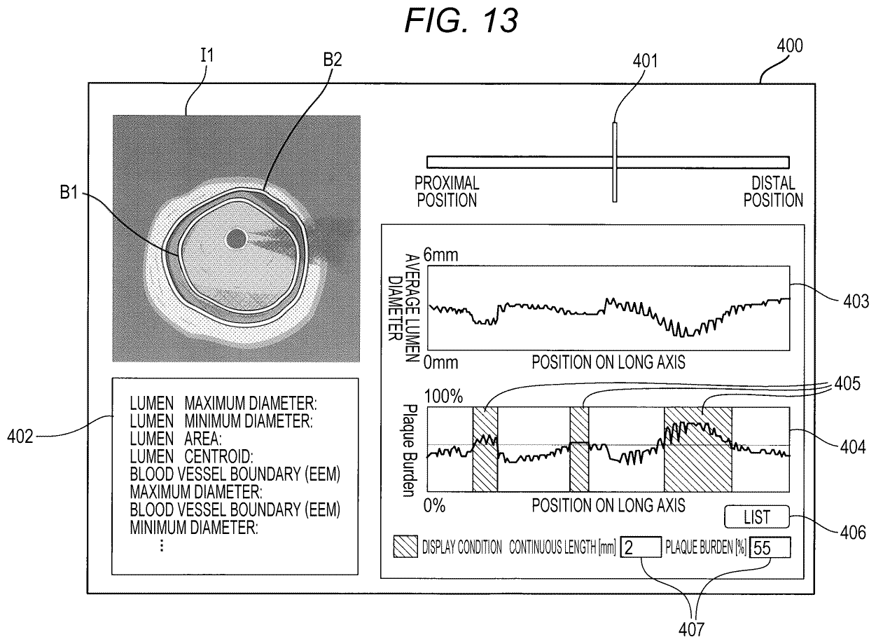

FIG. 13 illustrates an example of a screen 400 displayed on the display apparatus 4 in the second embodiment. The screen 400 of FIG. 13 is similar to the screen 400 illustrated in FIG. 8 in the first embodiment. In the configuration of the screen 400 illustrated in FIG. 13, the same reference signs are given to the configuration common to the screen 400 of the first embodiment, and the detailed description thereof is omitted.

Also in the second embodiment, a graph 404 illustrating the distribution of plaque burden with respect to the position on the longitudinal axis is displayed on the screen 400. In the graph 404, a graphic 405 is displayed in a superimposed manner in a range where the plaque burden is 55% or more. In the second embodiment, an edit box 407 that receives a setting change with respect to each of the threshold value of the ratio for displaying the graphic 405 and the threshold value of the length is displayed below the graph 404. In the edit box 407, a threshold value (=55%) of the ratio and a threshold value (=2 mm) of the length included in the setting data stored at that time are displayed. The inspection operator and the medical provider can change the threshold value of the ratio or the text of the threshold value of the length in the edit box 407 using the input apparatus 5. When the change has been made, the processing unit 30 of the image processing apparatus 3 determines in S121 that the changing operation has been performed (S121: YES).

As illustrated in FIG. 13, it is possible to receive a change in setting for each of data indicating different types of anatomical features.

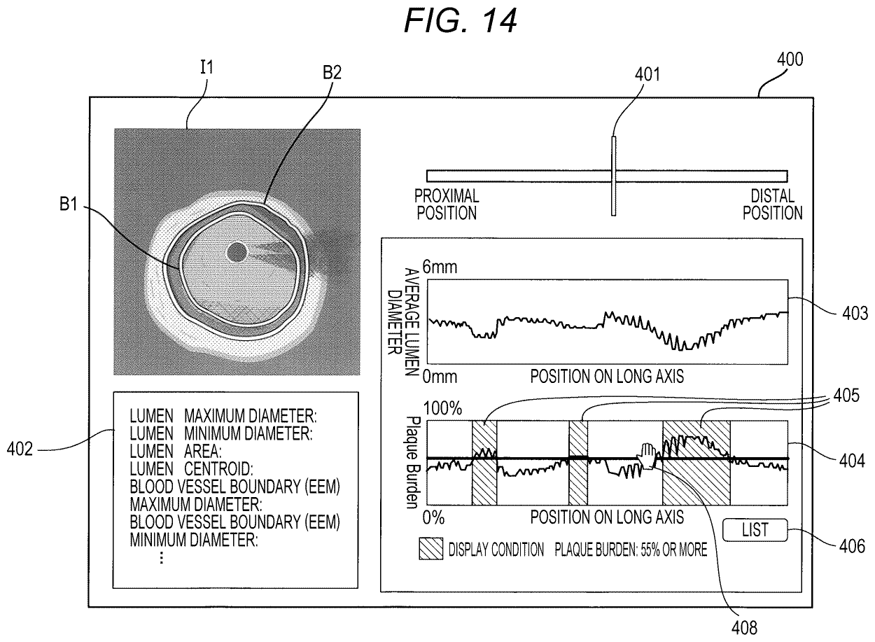

The method for receiving the change may be another method. FIG. 14 illustrates another example of the screen 400 displayed on the display apparatus 4 of the second embodiment. In FIG. 14, similarly to FIG. 13, the configurations common to those of the screen 400 of the first embodiment are denoted by the same reference signs, and detailed description thereof is omitted. On the screen 400 of FIG. 14, a straight line corresponding to the threshold value (=55%) of the ratio to the plaque burden is displayed in the graph 404 of the screen 400. In the screen 400 of FIG. 14, this straight line can be moved by the input apparatus 5. On the screen 400 of FIG. 14, a pointer 408 having a palm shape for holding a straight line is displayed. When the straight line is held by the pointer 408, the processing unit 30 determines that the changing operation has been performed (S121: YES), and updates the drawing of the graphic 405 according to the movement of the position of the pointer 408 (S111).

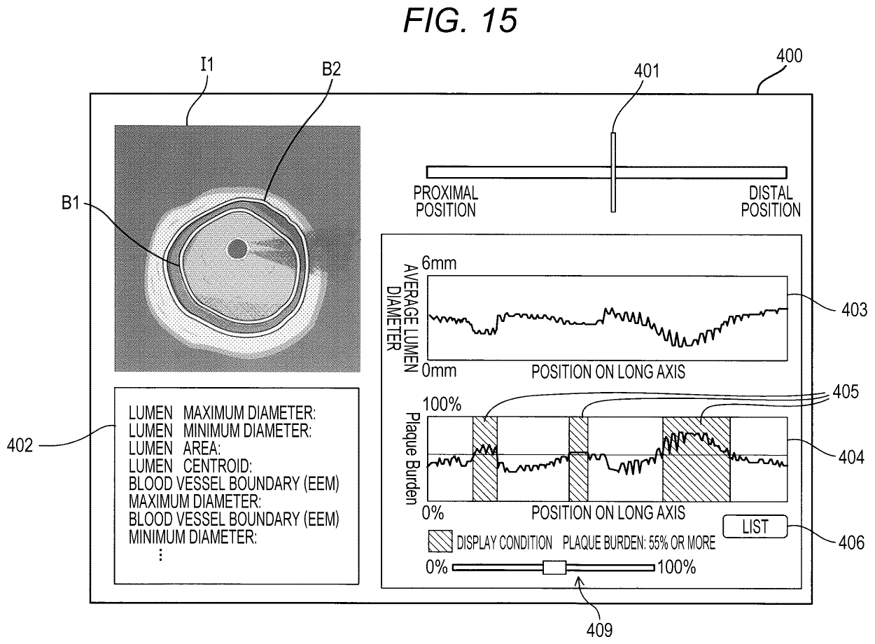

FIG. 15 illustrates another example of the screen 400 displayed on the display apparatus 4 of the second embodiment. In FIG. 15, similarly to FIG. 13, the configurations common to those of the screen 400 of the first embodiment are denoted by the same reference signs, and detailed description thereof is omitted. On the screen 400 of FIG. 15, a slide bar 409 that receives setting changes with respect to the threshold value of the ratio for displaying the graphic 405 and the threshold value of the length is displayed below the graph 404. The selector of the slide bar 409 can be moved by operating the input apparatus 5. When the selector is moved, the processing unit 30 determines that the changing operation has been performed (S121: YES), and updates the drawing of the graphic 405 according to the movement of the position of the selector (S111).

In the screen 400 of FIGS. 13 to 15, a mode for receiving the operation of changing the setting data for the graph 404 indicating the distribution of the plaque garden is illustrated. The present invention is not limited thereto, and an operation of changing the setting data may be received by a similar interface (e.g., the edit box 407, pointer 408, or slide bar 409) also for a distribution of data of other anatomical features, for example, a graph 403 of the distribution with respect to the position on the longitudinal axis of the average lumen diameter.

### Third Embodiment

In a third embodiment, when a graph of data indicating an anatomical feature is displayed, additional information is output on the graph. The configuration of the image diagnostic system 100 of the third embodiment is similar to the configuration of the image diagnostic system 100 of the first embodiment except for details of the process performed by the processing unit 30 of the image processing apparatus 3 described below and contents of a screen to be displayed. Therefore, among the configurations of the image diagnostic system 100 of the third embodiment, the configurations common to the image diagnostic system 100 of the first embodiment are denoted by the same reference signs, and a detailed description thereof will be omitted.

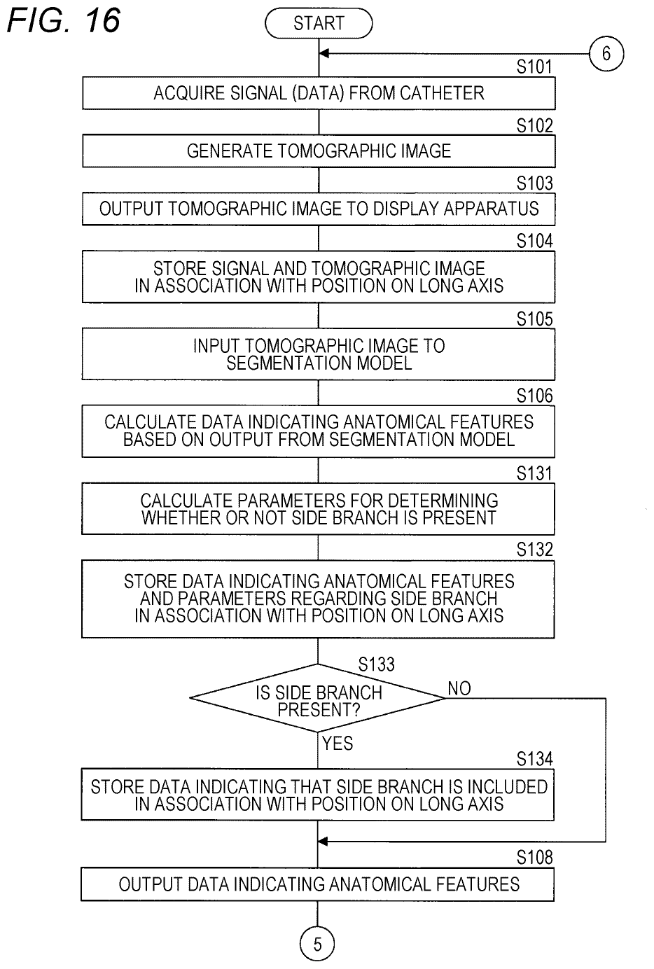

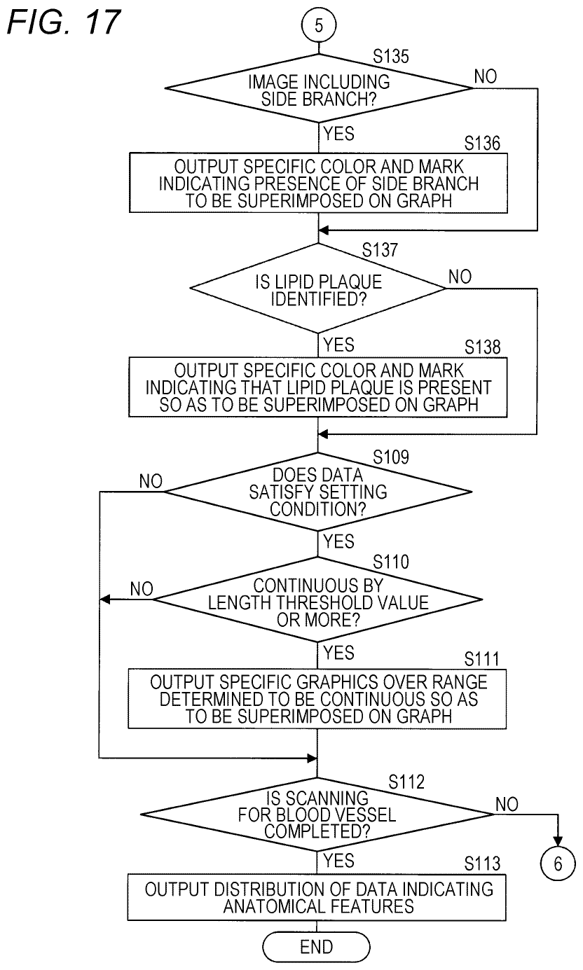

FIGS. 16 and 17 are flowcharts illustrating an example of the process performed by the image processing apparatus 3 according to the third embodiment. Among the steps illustrated in the flowcharts of FIGS. 16 and 17, the steps common to the processing procedures illustrated in the flowcharts of FIGS. 6 and 7 of the first embodiment are denoted by the same step numbers, and detailed description thereof is omitted.

In the third embodiment, after calculating the data indicating the anatomical features (S106), the processing unit 30 of the image processing apparatus 3 executes a step of calculating parameters for determining whether the side branch of the blood vessel is present in the tomographic image I1 based on the calculated parameters (S131).

Various methods can be considered for the step of side branch detection in S131. For example, the processing unit 30 calculates the parameters indicating the degree of deviation of the shape of the blood vessel boundary or the lumen boundary identified on the tomographic image I1 from the circular shape or the elliptical shape. In S131, for example, the processing unit 30 calculates the eccentricity obtained by dividing the difference between the maximum diameter and the minimum diameter of the diameter passing through the centroid of the inner region of the blood vessel boundary by the maximum diameter. The processing unit 30 may calculate circularity that is a ratio of the area of the region inside the blood vessel boundary and the circumferential length of the blood vessel boundary. In S131, for example, in a case where the tomographic image I1 (or the longitudinal image I2) and the image data of the lumen boundary and the blood vessel boundary are input, the processing unit 30 may use a model trained to output the accuracy with which the side branch is present. In S131, the processing unit 30 may calculate the degree of change when the diameter of the blood vessel boundary in the target tomographic image I1 is compared with the diameter of the blood vessel boundary at the position scanned so far.

The processing unit 30 stores the data indicating the anatomical features calculated in S106 and the parameters calculated for detection in S131 in association with the position on the longitudinal axis of the blood vessel (S132).

The processing unit 30 determines whether the target tomographic image I1 includes a side branch based on the calculated parameters (S133). When determining that the image includes the side branch (S133: YES), the processing unit 30 stores data indicating that the side branch is included in association with the position in the longitudinal direction (S134), and advances the process to S108. Before S134, a step of checking whether it is determined that a side branch is continuously present, or a step of calculating the angle of a side branch or the like may be performed.

In a case where it is determined in S133 that the image does not include a side branch (S133: NO), the processing unit 30 advances the process directly to S108.

When the data indicating the anatomical features is output as a graph (S108), the processing unit 30 determines whether the target tomographic image I1 is an image in which a side branch is present (S135). In S135, the processing unit 30 determines whether data indicating that a side branch is present is stored in association with the position on the longitudinal axis of the target tomographic image I1.

When it is determined that the image includes a side branch (S135: YES), the processing unit 30 outputs a graphic such as a specific color, a mark, or the like indicating the presence of the side branch to be superimposed on the graph (S136), and the process proceeds to S137.

When it is determined that the image does not include a side branch (S135: NO), the processing unit 30 advances the process directly to S137.

The processing unit 30 determines whether a lipid plaque is identified in the tomographic image I1 based on the tag image IS corresponding to the target tomographic image I1 (S137). When it is determined that the lipid plaque is identified (S137: YES), a specific color, mark, or the like indicating that the lipid plaque is present is output so as to be superimposed on the graph (S138), and the process proceeds to S109.

In S137, the processing unit 30 may determine that the lipid plaque is identified only when the lipid plaque is continuously identified not only by the target tomographic image I1 but also by the number corresponding to the length threshold value (for example, 2 mm).

When it is determined that the lipid plaque is not identified (S137: NO), the processing unit 30 advances the process directly to S109.

In this manner, the image processing apparatus 3 outputs additional information such as whether a side branch is present and whether a lipid plaque is present in association with the position on the longitudinal axis. As a result, in the distribution with respect to the average lumen diameter or the position on the longitudinal axis of plaque burden, the range of the lesion where plaque burden is equal to or more than the threshold value of the ratio, and the structure of the blood vessel around the lesion and the state of the lesion are output.

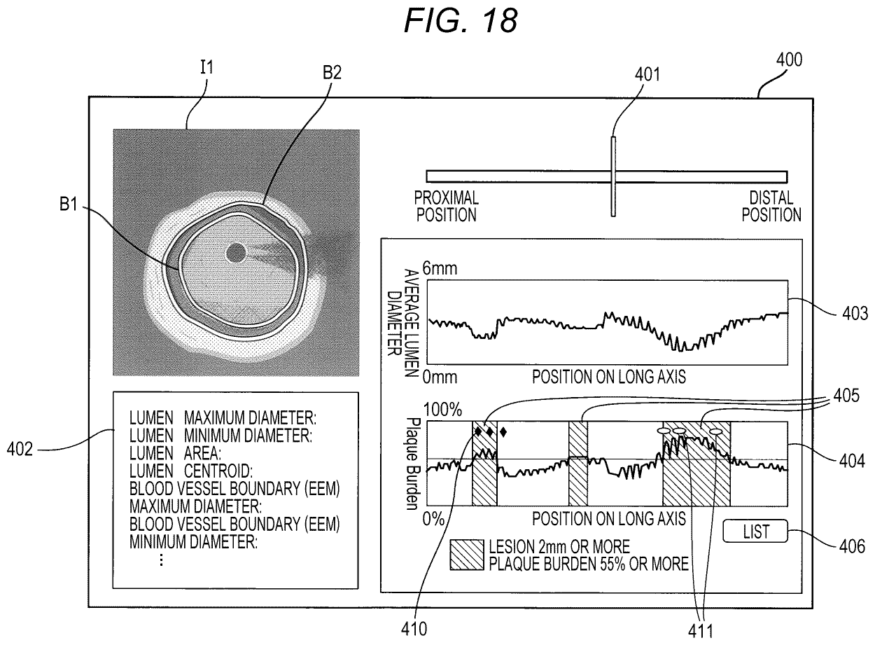

FIG. 18 illustrates an example of a screen 400 displayed on the display apparatus 4 in the third embodiment. The screen 400 of FIG. 18 is similar to the screen 400 illustrated in FIG. 8 in the first embodiment. In the configuration of the screen 400 illustrated in FIG. 18, the same reference signs are given to the configuration common to the screen 400 of the first embodiment, and the detailed description thereof is omitted.

Also in the third embodiment, a graph 404 illustrating the distribution of plaque burden with respect to the position on the longitudinal axis is displayed on the screen 400. In the graph 404, a graphic 405 is displayed in a superimposed manner in a range where the plaque burden is 55% or more. In the third embodiment, a black diamond-shaped mark 410 and a white elliptical mark 411 are displayed in a superimposed manner on the graph 404. The diamond-shaped mark 410 is a mark indicating that a side branch is present, and the elliptical mark 411 is a mark indicating that a lipid plaque is identified.

By the process of the image processing apparatus 3 of the third embodiment, as in the screen 400 illustrated in FIG. 18, marks 410 and 411 indicating the detected additional information is further displayed in a superimposed manner on the graphic 405 indicating that the plaque burden is 55% or more. The inspection operator and other medical workers who visually recognize the graph 404 on which the marks 410 and 411 and the graphic 405 are superimposed can grasp that the plaque burden is 55% or more in the blood vessel in the range where the graphic 405 is displayed, and can also recognize a portion where there is a possibility of blocking the side branch when the stent is placed. This makes it possible to more accurately determine how to select a stent and a balloon for expanding the range in which the plaque burden is present by the length threshold value or more.

It is a matter of course that the marks 410 and 411 may be superimposed and displayed on the graph 403 illustrating the distribution of the average lumen diameter with respect to the position in the longitudinal direction. In this case, the inspection operator and the other medical workers can grasp that the average lumen diameter is less than the threshold value of the lumen diameter in the blood vessel in the range in which the graphic 405 is displayed, and can recognize a portion where the lipid plaque exists and the stability is insufficient in order to place the stent.

### Fourth Embodiment

In a fourth embodiment, the imaging device 11 is a dual type catheter device including a transmitter and a receiver of waves of different wavelengths (e.g., an ultrasonic wave and light), respectively. The imaging device 11 includes an ultrasonic probe including an IVUS ultrasonic transducer and an ultrasonic sensor, and an OFDI device including a near-infrared laser, a near-infrared sensor, and the like. The OFDI device is a device including an optical element having a lens function and a reflection function at the distal end, and may have a structure of guiding light to a near-infrared laser and a near-infrared sensor connected via an optical fiber. The dual type target is not limited to a combination of IVUS and OFDI, and may be echo or the like.

The image processing apparatus 3 according to the fourth embodiment acquires the IVUS and OFDI signals for each scan output from the imaging device 11 of the catheter 1, and generates a plurality of IVUS medical images and a plurality of OFDI medical images in the longitudinal direction. The image processing apparatus 3 analyzes and processes the bifurcation structure of the blood vessel based on the medical image (i.e., the tomographic image and/or longitudinal image) obtained for each of IVUS and OFDI, processes the medical image for easy viewing, and outputs the medical image so as to be visually recognizable by a doctor, an inspection operator, or other medical workers.

The configurations of the image diagnostic system 100 and the image processing apparatus 3 in the fourth embodiment are similar to the configurations of the image diagnostic system 100 and the image processing apparatus 3 in the first embodiment except that the above-described imaging device 11 is of a dual type and a part of the processing procedure by the image processing apparatus 3 associated therewith. Therefore, regarding the configuration of the image processing apparatus 3, the same reference signs are given to the configuration common to the configuration of the image processing apparatus 3 of the first embodiment, and a detailed description thereof will be omitted.

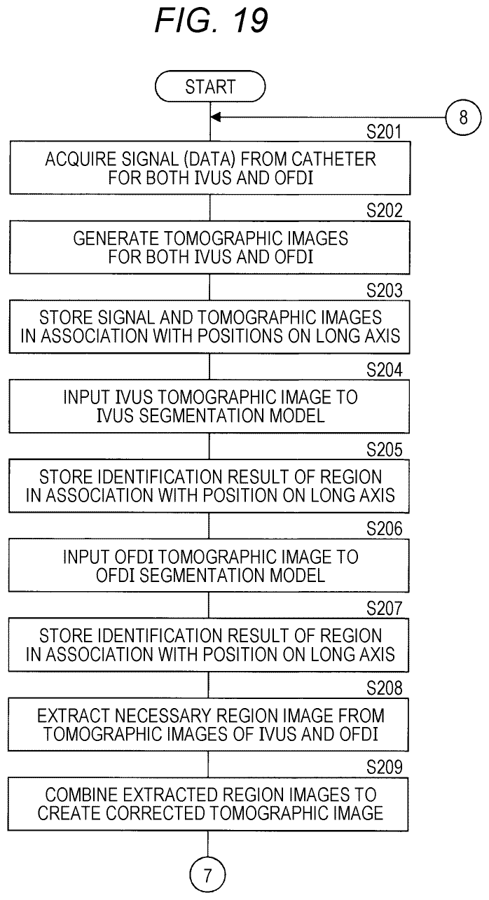

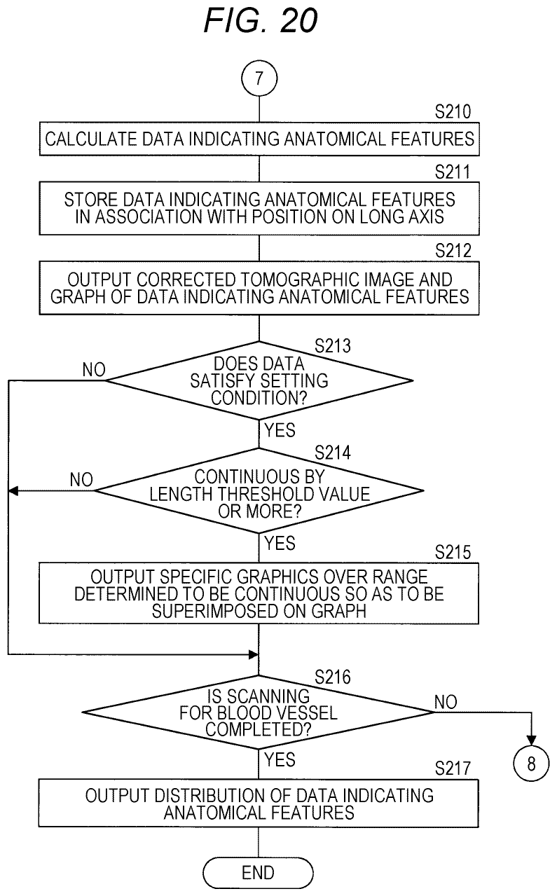

FIGS. 19 and 20 are flowcharts illustrating an example of the process performed by the image processing apparatus 3 according to the fourth embodiment. When a signal is output from the imaging device 11 of the catheter 1, the processing unit 30 of the image processing apparatus 3 starts the following process.

The processing unit 30 generates tomographic images I11 and I12 (S202) each time signals from the imaging device 11 of the catheter 1 are acquired for a predetermined amount (for example, 360°) for both IVUS and OFDI (S201). In S202, the processing unit 30 performs the polar coordinate transformation (e.g., inverse transformation) on the signals arranged in the rectangle for each of IVUS and OFDI to generate the tomographic images I11 and I12.

For each of IVUS and OFDI, the processing unit 30 stores the signal data acquired in S201 and the tomographic images I11 and I12 generated in S202 in the storage unit 31 in association with the positions on the longitudinal axis of the blood vessel (S203).

The processing unit 30 inputs the IVUS tomographic image I11 to the segmentation model 31M for IVUS (S204). The processing unit 30 stores the identification result (i.e., the tag image IS1) of the region output from the segmentation model 31M for IVUS in the storage unit 31 in association with the position on the longitudinal axis of the blood vessel (S205).

The processing unit 30 inputs the OFDI tomographic image I12 to the segmentation model 31M for OFDI (S206). The processing unit 30 stores the region identification result (i.e., the tag image IS2) output from the segmentation model 31M for OFDI in the storage unit 31 in association with the position on the longitudinal axis of the blood vessel (S207).

The processing unit 30 extracts a necessary region image from the tomographic image I11 and the tomographic image I12 based on the region identification result (i.e., the tag image IS1) for the tomographic image I11 of IVUS and the region identification result (i.e., the tag image IS2) for the tomographic image I12 of OFDI (S208). In S208, the processing unit 30 extracts, for example, a region image of a membrane range and a region image of a lipid plaque range respectively corresponding to the media range and the adventitia range from the IVUS tomographic image I11, and extracts a region image of a lumen range and a region image of a fibrous plaque and calcified plaque range from the OFDI tomographic image I12. That is, the processing unit 30 appropriately extracts the anatomical features and the range of the lesion where the identification of the range is clear in each of IVUS and OFDI.

The processing unit 30 combines the extracted region images to create a corrected tomographic image (S209).

The processing unit 30 calculates, with respect to the corrected tomographic image, data indicating anatomical features including the maximum value, the minimum diameter, the average inner diameter, and the plaque burden in the range inside from the lumen boundary of the blood vessel (S210).

The processing unit 30 stores the data (e.g., the average inner diameter inside the lumen boundary, plaque burden, and the like) indicating the anatomical features calculated in S210 in the storage unit 31 in association with the position on the longitudinal axis of the blood vessel (S211).

The processing unit 30 outputs the corrected tomographic image created in S209 and the graph of data indicating the anatomical features calculated in S210 to the screen being displayed on the display apparatus 4 (S212). In S212, the processing unit 30 outputs a graph indicating a transition of data indicating anatomical features with respect to the longitudinal direction. In S212, the processing unit 30 may also display the numerical value itself of the data.

The processing unit 30 determines whether the data indicating the anatomical features calculated in step 210 satisfies a setting condition included in the setting data stored in the storage unit 31 (S213). When it is determined that the data indicating the anatomical features satisfies the setting condition (S213: YES), the processing unit 30 determines whether the data determined to satisfy the setting condition is continuous in the longitudinal direction by a length threshold value or more included in the setting data (S214).

When it is determined in S214 that the data is continuous (S214: YES), the processing unit 30 outputs graphics of specific colors, patterns, and the like over the range of positions on the longitudinal axis determined to be continuous so as to be superimposed on the graph output in S212 (S215).

The processing unit 30 determines whether scanning of the catheter 1 by the imaging device 11 has been completed (S216). When it is determined that the scanning is not completed (S216: YES), the processing unit 30 returns the process to S201 and generates the next tomographic images I11 and I12.

When it is determined that the scanning has been completed (S216: YES), the processing unit 30 outputs again the distribution of data indicating the anatomical features with respect to the entire scanned blood vessel in the longitudinal direction (S217), and ends the process.

When it is determined in S213 that the setting condition is not satisfied (S213: NO), the processing unit 30 advances the process directly to S216. When it is determined in S214 that the data does not continue for the length threshold value or more (S214: NO), the processing unit 30 advances the process directly to S216.

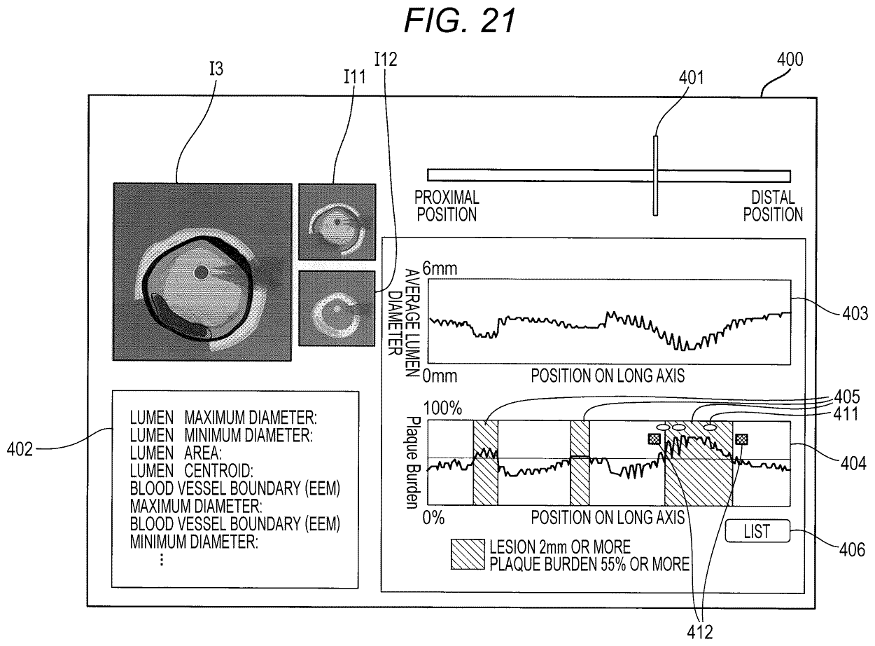

The information output by the process illustrated in FIGS. 19 and 20 will be described with a specific example. FIG. 21 illustrates an example of the screen 400 displayed on the display apparatus 4. The screen 400 of FIG. 21 is similar to the screen 400 illustrated in FIG. 8 in the first embodiment. In the configuration of the screen 400 illustrated in FIG. 21, the same reference signs are given to the configuration common to the screen 400 of the first embodiment, and the detailed description thereof is omitted.

The screen 400 output by the image processing apparatus 3 in the fourth embodiment includes a cursor 401 indicating the position on the longitudinal axis of the blood vessel corresponding to the displayed corrected tomographic image I3, and tomographic images I11 and I12 and the corrected tomographic image I3 generated based on the signal obtained at the position. The screen 400 includes a data field 402 that displays numerical values of data indicating the anatomical features calculated by the image processing on the corrected tomographic image I3.

The screen 400 in FIG. 21 also includes graphs 403 and 404 illustrating the distribution of data indicating anatomical features with respect to positions on the longitudinal axis of the blood vessel. The graph 403 illustrates the distribution of the average lumen diameter with respect to the position on the longitudinal axis, and the graph 404 illustrates the distribution of the plaque burden with respect to the position on the longitudinal axis. A graphic 405 is superimposed and displayed on the graph 404. The graphic 405 indicates a range in which a portion where the plaque burden is equal to or more than the threshold value of the ratio (here, 55%) is continuous in the longitudinal direction by 2 mm or more of the threshold value of the length. This makes it possible to more accurately determine which size should be selected as a stent for expanding the high plaque burden range and how to select a balloon for placing the stent.

On the screen 400 in FIG. 21, not only a mark 411 indicating that a lipid plaque is present but also a mark 412 indicating a position where a fibrous plaque or a calcified plaque is present are displayed. In the fourth embodiment, since not only IVUS but also the tomographic image I12 based on OFDI is used as the dual imaging device 11, it is possible to more accurately grasp the situation by displaying the mark 412 indicating the presence of fibrous plaque and calcified plaque with an ambiguous boundary from the tomographic image I11 of IVUS together.

In the fourth embodiment, the corrected tomographic image I3 is displayed, and it is possible to accurately grasp even a lesion that is difficult to interpret with only one of the tomographic image I11 of IVUS and the tomographic image I12 of OFDI. In addition, in the fourth embodiment, data indicating anatomical feature data is calculated and graphed from the corrected tomographic image I3 obtained by extracting regions that are easily identified from each of the tomographic image I11 of IVUS and the tomographic image I12 of OFDI. As a result, the information obtained from the output graph becomes more accurate, and the medical provider can more quickly and more accurately determine what stent or balloon should be used regardless of the experience of image interpretation.

The embodiments described above are illustrative in all respects and are not restrictive. The scope of the present invention is defined by the claims, and includes meanings equivalent to the claims and all modifications within the scope.

What is claimed is:

1. An image diagnostic system comprising:
a catheter insertable into a luminal organ and including a plurality of imaging devices;
a display;
a memory; and
a processor configured to execute a program that is stored in the memory to perform the steps of:
generating tomographic images of the luminal organ based on signals that are output from the imaging devices,
combining the tomographic images to generate a first tomographic image,
calculating one or more distributions of values each indicating an anatomical feature of the luminal organ from the first tomographic image,
selecting at least one of the distributions, and specifying one or more first portions of the luminal organ that are located along a longitudinal direction of the luminal organ and at which the distribution of values of the selected distribution meets one or more conditions, and
controlling the display to display:
one or more graphs each illustrating a corresponding one of the calculated distributions, and
one or more first graphics on the graphs at locations corresponding to the specified first portions.

2. The image diagnostic system according to claim 1, wherein
the conditions are met when a predetermined number of values exceeds a threshold value, the predetermined number corresponding to a length of the first portions.

3. The image diagnostic system according to claim 1, wherein
the conditions include a plurality of sets of conditions for said one of the distributions, and
the steps include:
specifying a plurality of sets of one or more second portions in the longitudinal direction of the luminal organ in which said one of the distributions meets the respective sets of conditions, and
controlling the display to display:
a list of graphs each illustrating said one of the distributions, a total number of the graphs corresponding to a total number of the sets of conditions, and
a set of one or more second graphics on each of the graphs corresponding to one of the sets of the second portions.

4. The image diagnostic system according to claim 1, wherein
the luminal organ is a blood vessel, and
each of the values that exceed a threshold value indicates a ratio of an area of plaque to a membrane area inside a part of the blood vessel.

5. The image diagnostic system according to claim 1, wherein
each of the values that are less than a threshold value indicates an average lumen diameter of a part of the luminal organ.

6. The image diagnostic system according to claim 1, wherein
the luminal organ is a blood vessel,
said calculating includes:
calculating a first distribution of first values each indicating a ratio of an area of plaque to a membrane area inside a part of the blood vessel, and
calculating a second distribution of second values each indicating an average lumen diameter of a part of the blood vessel,
said specifying includes specifying:
one or more portions of the blood vessel at which locations the first values exceed a first threshold value, and
one or more portions of the blood vessel at which locations the second values are less than a second threshold value, and
said controlling includes controlling the display to display:
a first graph illustrating the distribution of the first values,
a second graph illustrating the distribution of the second values,
one or more graphics on the first graph at locations of the first graph corresponding to the portions at which the first values exceed the first threshold value, and
one or more graphics on the second graph at locations of the second graph corresponding to the portions at which the second values are less than the second threshold value.

7. The image diagnostic system according to claim 1, wherein
the steps further include:
specifying a lipid plaque in the first tomographic image, and
said controlling includes controlling the display to display a second graphic at a location of the display where the lipid plaque is specified.

8. The image diagnostic system according to claim 1, wherein
the steps further include:
specifying a side branch of the luminal organ in the first tomographic image, and
said controlling includes controlling the display to display a second graphic at a location of the display where the side branch is specified.

9. The image diagnostic system according to claim 1, wherein
said controlling includes controlling the display to display one or more graphical user interface (GUI) components through which one or more values related to the conditions can be input.

10. The image diagnostic system according to claim 9, wherein
the values that can be input are threshold values to be compared to the values indicating the anatomical feature of the luminal organ.

11. The image diagnostic system according to claim 9, wherein
one of the GUI components is a slide bar displayed adjacent to one of the graphs.

12. An image diagnostic method performed by a system that includes a catheter insertable into a luminal organ and including a plurality of imaging devices, the image diagnostic method comprising:
generating tomographic images of the luminal organ based on signals that are output from the imaging devices;
combining the tomographic images to generate a first tomographic image;
calculating one or more distributions of values each indicating an anatomical feature of the luminal organ from the first tomographic image;
selecting at least one of the distributions, and specifying one or more first portions of the luminal organ that are located along a longitudinal direction of the luminal organ and at which the distribution of values of the selected distribution meets one or more conditions; and
displaying:
one or more graphs each illustrating a corresponding one of the calculated distributions, and
one or more first graphics on the graphs at locations corresponding to the specified first portions.

13. The image diagnostic method according to claim 12, wherein
the conditions are met when a predetermined number of values exceeds a threshold value, the predetermined number corresponding to a length of the first portions.

14. The image diagnostic method according to claim 12, wherein
the conditions include a plurality of sets of conditions for said one of the distributions, and
the image diagnostic method further comprises:
specifying a plurality of sets of one or more second portions in the longitudinal direction of the luminal organ in which said one of the distributions meets the respective sets of conditions, and
displaying:
a list of graphs each illustrating said one of the distributions, a total number of the graphs corresponding to a total number of the sets of conditions, and
a set of one or more second graphics on each of the graphs corresponding to one of the sets of the second portions.

15. The image diagnostic method according to claim 12, wherein
the luminal organ is a blood vessel, and
each of the values that exceed a threshold value indicates a ratio of an area of plaque to a membrane area inside a part of the blood vessel.

16. The image diagnostic method according to claim 12, wherein
each of the values that are less than a threshold value indicates an average lumen diameter of a part of the luminal organ.

17. The image diagnostic method according to claim 12, wherein
the luminal organ is a blood vessel,
said calculating includes:
calculating a first distribution of first values each indicating a ratio of an area of plaque to a membrane area inside a part of the blood vessel, and
calculating a second distribution of second values each indicating an average lumen diameter of a part of the blood vessel,
said specifying includes specifying:
one or more portions of the blood vessel at which locations the first values exceed a first threshold value, and
one or more portions of the blood vessel at which locations the second values are less than a second threshold value, and
said displaying includes displaying:
a first graph illustrating the distribution of the first values,
a second graph illustrating the distribution of the second values,
one or more graphics on the first graph at locations of the first graph corresponding to the portions at which the first values exceed the first threshold value, and
one or more graphics on the second graph at locations of the second graph corresponding to the portions at which the second values are less than the second threshold value.

18. The image diagnostic method according to claim 12, further comprising:
specifying a lipid plaque in the first tomographic image; and
displaying a second graphic at a location of a display where the lipid plaque is specified.

19. The image diagnostic method according to claim 12, further comprising:
specifying a side branch of the luminal organ in the first tomographic image; and
displaying a second graphic at a location of a display where the side branch is specified.

20. A non-transitory computer readable storage medium storing a program causing a computer to execute an image diagnostic method comprising:
generating tomographic images of a luminal organ based on signals that are output from a plurality of imaging devices of a catheter;
combining the tomographic images to generate a first tomographic image;
calculating one or more distributions of values each indicating an anatomical feature of the luminal organ from the first tomographic image;
selecting at least one of the distributions, and specifying one or more first portions of the luminal organ that are located along a longitudinal direction of the luminal organ and at which the distribution of values of the selected distribution meets one or more conditions; and
displaying:
one or more graphs each illustrating a corresponding one of the calculated distributions, and
one or more first graphics on the graphs at locations corresponding to the specified first portions.
